(19) United States
(12) Patent Application Publication
(10) Pub. No.: US 2009/0259300 A1
(43) Pub. Date: Oct. 15, 2009

(54) Medical Devices With an Interlocking Coating and Methods of Making the Same

(75) Inventors: William E. Dorogy, JR., Newburyport, MA (US); Raed Rizq, Fridley, MN (US); Ben Arcand, Minneapolis, MN (US); Steve Kangas, Woodbury, MN (US); Jan Weber, Maastricht (NL)

Correspondence Address: FISH & RICHARDSON PC, P.O. BOX 1022, MINNEAPOLIS, MN, 55440-1022, US

(73) Assignee: BOSTON SCIENTIFIC SCIMED, INC., Maple Grove, MN (US)

(21) Appl. No.: 12/421,107

(22) Filed: Apr. 9, 2009

Related U.S. Application Data
(60) Provisional application No. 61/123,614, filed on Apr. 10, 2008.

(51) Int. Cl. A61F 2/06

(52) U.S. Cl. 623/1.36; 623/1.42; 623/1.46

(57) ABSTRACT

Disclosed herein are medical devices, such as intravascular stents, for delivering a therapeutic agent to the body tissue of a patient, and a method for making such medical devices. More particularly, the medical devices have a coating that includes a polymer that adheres to the surface of the medical device so that the coating is able to resist damage during loading, deployment and implantation.

### 1.0 INTRODUCTION

The medical devices described herein, which include intravascular stents, are capable of delivering a therapeutic agent to the body tissue of a patient. Also described herein are methods for making such medical devices. More particularly, the medical devices have a coating that includes a polymer that adheres to the surface of the medical device so that the coating is able to resist damage during loading deployment and implantation.

### 2.0 BACKGROUND

Many implantable medical devices, such as intravascular stents, have a drug-releasing coating. Such coatings usually include a polymer and a therapeutic agent. However, certain polymer coatings have shown poor adhesion to the medical device. This poor adhesion makes the coatings susceptible to deformation and damage during loading, deployment and implantation of the medical device. Any damage to the polymer coating may not only alter the release profile of the therapeutic agent, leading to an undesirable increase or decrease in the therapeutic agent release rate, but damage to the coating may also result in delamination, flaking, peeling, or cracking of the coating, especially during deployment. Such damage to the coating can result in injury caused by detached debris being released into the bloodstream.

For instance, balloon expandable stents must be put in an unexpanded or “crimped” state before being delivered to a body lumen. During the crimping process, coated stent struts are placed in contact With each other and can possibly stick to each other. When the stent is expanded or uncrimped, the coating on the struts that have stuck to each other can be damaged, torn off or otherwise removed. Moreover, if the polymer coating is applied to the inner surface of the stent, it may stick to the balloon used to expand the stent ashen the balloon contacts the inner surface of the stent during expansion. Such contact with the balloon may prevent a successful deployment of the medical device, as well as, damage the polymer coating.

Similar to balloon-expandable stents, polymer coatings on self-expanding stents can also interfere with the delivery of the stent. Self-expanding stents are usually delivered using a pull-back sheath system. When the system is activated to deliver the stent, the sheath is pulled back, exposing the stent and allowing the stent to expand itself. As the sheath is pulled back it slides over the outer surface of the stent. Polymer coatings located on the outer or abluminal surface of the stent can stick to the sheath as it is being pulled back and disrupt the delivery of the stent as well as any therapeutic agent disposed on the stent.

One possible solution is to apply a primer coating to the surface of a medical device to improve the adherence of the coating to the medical device. U.S. Pat. No. 7,001,421 to Cheng et al., which is incorporated herein by reference, suggests applying a primer coating of phenoxy resin onto a stent to provide a substrate onto which the polymer coating can adhere. However, such phenoxy resin coatings have certain disadvantages. Thus, there is a need for an improved method for adhering a polymer coating onto a medical device.

Accordingly, there is a need for a medical device that includes a coating that adheres to the medical device such that the coating is able to resist damage during loading, deployment and implantation of the medical device and is also capable of delivering a desired amount of a therapeutic agent. Furthermore, there is a need for a method of making such medical devices.

### 3.0 SUMMARY

The embodiments described herein are directed to a medical device, preferably an intravascular stent that has a coating that adheres to the surface of the medical device and is able to resist damage during loading, deployment and implantation of the medical device while also delivering a desired amount of a therapeutic agent. In certain embodiments, the coatings include a polymer that adheres to the surface of a medical device. In other embodiments, the coatings include a polymer that adheres to a first coating material disposed on the surface of a medical device.

In one embodiment, the medical device is an implantable stent comprising a substrate having a surface. A coating is disposed on at least a portion of the surface. The coating includes a first coating material having a surface. The coating material includes a metal or a metal-containing compound, e.g. a metal carbide, a metal nitride or a metal oxide, having a plurality of pores therein. At least some of the pores are in fluid communication with the first coating material surface. The coating also includes a second coating material disposed on at least a portion of the first coating material surface and in at least some of the pores, forming an interlock between the second coating material and the substrate. The second coating material includes a first polymer and a first therapeutic agent. The average peel strength of the second coating material from the stent is about 250 grams per inch width or greater. In some embodiments the average peel strength is about 1000 grams per inch width or greater. In other embodiments the average peel strength is about 1000 grams per inch width to about 3000 grams per inch width.

In another embodiment, the medical device can be an implantable stent comprising a substrate having a surface. The substrate includes a metal or a metal-containing compound having a plurality of pores therein, and at least some of the pores are in fluid communication with the substrate surface. A coating comprising a coating material is disposed on at least a portion of the substrate surface and in at least some of the pores, forming an interlock between the coating material and the substrate. The coating material includes a first polymer and a first therapeutic agent. In some embodiments, the average peel strength of the coating material from the stent is 250 grams per inch width or greater. In other embodiments, the average peel strength of the coating material from the stent is 1000 grams per inch width or greater. In still other embodiment, the average peel strength is about 1000 grams per inch width to about 3000 grams per inch width.

Furthermore, in another embodiment, the medical device is an implantable stent comprising a substrate having a surface. A coating is disposed on at least a portion of the substrate surface, which includes a first coating material. This first coating material has a surface and includes a metal or a metal-containing compound having a plurality of pores therein. At least some of the pores are in fluid communication with the first coating material surface. The coating also includes a second coating material disposed on at least a portion of the first coating material surface and in at least some of the pores, forming an interlock between the coating and the substrate. The second coating material comprises a first composition that comprises a first polymer and that is substantially free of a therapeutic agent. The second coating material also comprises a second composition that comprises a second polymer and a therapeutic agent.

In yet another embodiment, the medical device is an implantable stent comprising a substrate having a surface, wherein the substrate includes a metal or a metal-containing compound having a plurality of pores therein. At least some of the pores are in fluid communication with the substrate surface. A coating comprising a coating material is disposed on at least a portion of the substrate surface and in at least some of the pores, forming an interlock between the coating material and the substrate. The coating material comprises a first composition that comprises a first polymer and that is substantially free of a therapeutic agent, and a second composition that comprises a second polymer and a therapeutic agent.

### 4.0 DEFINITIONS

As used herein, the term “interlock” refers to a connection between a material and itself or two or more materials so that separation or movement between the material(s) is constrained. For example, a connection can include the interface of the geometry of one material with itself or the geometry of another material such that one or both materials must be deformed, altered or broken to be disconnected. In certain embodiments, interlock refers to adhesion between two or more materials, such as a polymer and a medical device substrate. In other embodiments, interlock refers to a connection between one part of a material and another part of the material.

As used herein, the phrases “average peel strength” or “average peel force” refers to the average amount of force per unit distance needed to remove an inch width of coating material from a surface or other material upon which the coating material is disposed.

As used herein, the term “substantially free of a coating or coating composition” refers to not having a coating or coating material intentionally disposed thereon.

As used herein, the term “substantially free of a therapeutic agent” refers to not intentionally including a therapeutic agent.

As used herein, the term “about” is synonymous with the term “approximately,” and refers to a little more or less than the stated value.

### 5.0 BRIEF DESCRIPTION OF THE DRAWINGS

FIG. 1 shows a cross-sectional view of an example of a medical device having a coating comprising two coating materials.

FIG. 2 shows a cross-sectional view of an embodiment of a medical device having a coating comprising a coating material.

FIG. 3 shows a cross-sectional view of another embodiment of a medical device having a coating comprising two coating materials.

FIG. 4 shows a cross-sectional view of yet another embodiment of a medical device having a coating comprising two coating materials.

FIG. 5 shows a cross-sectional view of an embodiment of a medical device having a coating comprising a coating material.

FIG. 6 shows a cross-sectional view of another embodiment of a medical device having a coating comprising a coating material.

FIG. 7 shows a perspective view of an example of a stent having a sidewall with openings therein.

FIG. 8 is a scanning electron micrograph of a section of a polymer-coated porous stainless steel substrate.

FIG. 9 is another scanning electron micrograph of a section of a polymer-coated porous stainless steel substrate.

FIG. 10 is a bar graph depicting the percent in peel adhesion of different polymer coatings from a substrate.

### 6.0 DETAILED DESCRIPTION

### 6.1 Coated Medical Devices

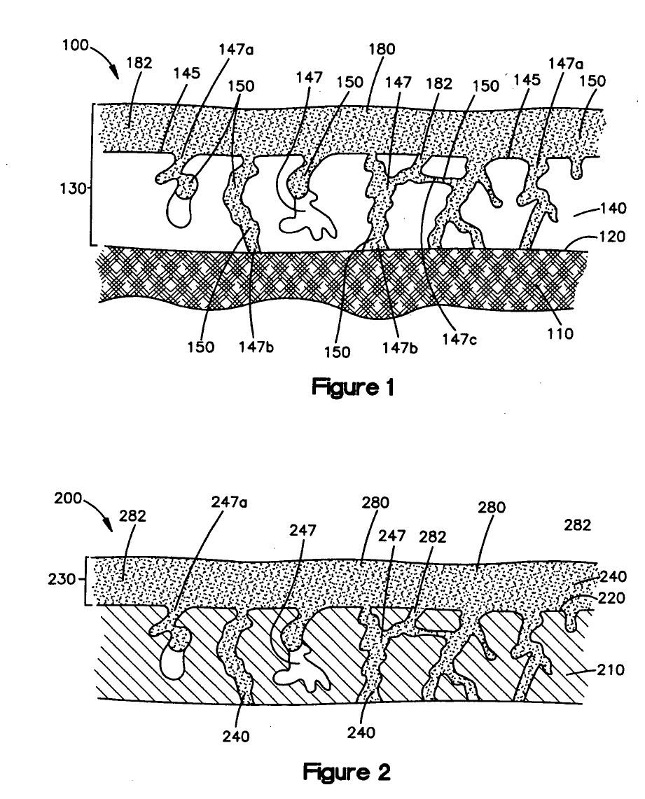

FIG. 1 shows a cross-sectional view of an embodiment of a coating disposed on a surface of a medical device, such as an intravascular stent. In this embodiment, the coating includes a porous first coating material disposed on the surface of a medical device and a second coating material disposed on the porous first coating material. The medical device 100 has a substrate 110 having a surface 120. A coating 130, which comprises a first coating material 140 and a second coating material 150, is disposed on at least a portion of the substrate surface 120. The first coating material 140 has a surface 145 and a plurality of pores 147 in the first coating material 140. At least some of the pores 147a are in fluid communication with the surface of the first coating material 145. The first coating material 140 can include a metal or a metal-containing compound such as a metal carbide, a metal nitride or a metal oxide. The second coating material 150, which includes a first polymer 180 and a therapeutic agent 182, is disposed on at least a portion of the first coating material 140 and within at least one of the pores 147. In this embodiment, the presence of the second coating material 150 on the first coating material 140 and in the pores 147 forms an interlock between the second coating material 150 and the stent substrate 120. In some embodiments, the second coating material can include more than one polymer or more than one therapeutic agent.

The strength of the interlock between materials, such as a coating and a stent substrate can be determined by measuring the peal strength of the coating. The unit of measure of the peel strength is the peel force (g/in) required to peel the coating divided by the line defined by the intersection of the peeled portion of the coating and the unpeeled portion of the coating that is still on the substrate. For example, the force required to peel the coating from a strut might be estimated to be the peel force multiplied by the width of the strut in inches.

In certain embodiments, such as the one shown in FIG. 1, the average peel strength of the second coating material from the stent can be greater than about 250 grams per inch width; greater than about 500 grams per inch width; greater than about 750 grams per inch width; or greater than about 1000 grams per inch width. For example, the average peel strength can be about 250 grams per inch width to about 3000 grams per inch width; about 500 grams per inch width to about 3000 grams per inch width; or about 1000 grams per inch width to about 3000 grams per inch width.

The peel strength of a coating can be determined by fixing a coated substrate on a tensile tester. The coated substrate is prepared such that a portion of the coating can be easily peeled or removed from the substrate without damaging the coating. A portion of the coating, e.g. coating flap, is carefully pulled back and secured to a clamp located on a moveable cross-head of the tensile tester. The coating flap is peeled back at a set speed, such as 6″/min., at about 180° peel angle. Peel strength is then measured as a function of cross-head displacement. Testing is continued until the peel strength values either remain constant or significantly decrease.

In some embodiments, such as the one shown in FIG. 1 and the others described herein, at least some of the pores 147b extend from the first coating material surface 145 to the substrate surface 120 and the second coating material 150 in at least some of the pores 147b contacts the substrate surface 120. Also, as shown in FIG. 1, in some embodiments at least some of the pores 147c can be interconnected thus creating an interlock between the polymer in one pore and the polymer in a another pore.

In certain embodiments, such as those described herein, the average pore size can range from about 0.1 nm to about 300 μm, about 1 nm to about 100 μm, about 10 nm to about 50 μm, about 50 nm to about 10 μm, or about 100 nm to about 10 μm. In certain embodiments, the average pore size can be about 0.2 μm, about 0.5 μm or about 1 μm. Also, in certain embodiments, such as those described herein, the porosity of the coating materials or medical device substrates in which the pores or interstices are disposed, i.e. the ratio of the volume of pores or interstices in the coating or substrate material to the volume of such material, can be about 10%, 20%, 30%, 40%, 50%, 60%, 70%, 80% or 90%. In certain embodiments, the porosity can be about 50%. Pore size and porosity can be measured by any means known in the art including, but not limited to, mercury porosimetry and nitrogen absorption.

In the embodiments in which the medical device is a stent, the coating can be disposed on the abluminal surface, i.e., the surface that faces away from the lumen of the stent. In some embodiments, the luminal surface of the stent, i.e., the surface that faces the lumen of the stent, is substantially free of the coating. In other embodiments, the coating can be disposed on the luminal surface while the abluminal surface is substantially free of the coating. In yet other embodiments, a coating is disposed on the abluminal surface and a coating is disposed on the luminal surface. When a coating is disposed on both the abluminal and luminal surfaces of a stent, the coating disposed on the abluminal surface can be the same or different than the coating disposed on the luminal surface of the stent.

FIG. 2 shows a cross-sectional view of another embodiment of a coated medical device, such as a stent. In this embodiment, the substrate of the medical device comprises a plurality of pores. A coating comprising a coating material is disposed on the surface of the medical device and in the pores. More specifically, the medical device 200 has a substrate 210 and a surface 220. The substrate 210 comprises a plurality of pores 247. At least some of the pores 247a are in fluid communication with the substrate surface 220. Also, a coating 230, which comprises a coating material 240, is disposed on the substrate surface 220 and in at least some of the pores 247. The substrate 210 can include a metal or a metal-containing compound such as a metal carbide, a metal nitride or a metal oxide. In this embodiment, the coating material 240 includes a first polymer 280 and a therapeutic agent 282. In this embodiment, the presence of the coating material 240 on the substrate surface 220 and in the pores 247 forms an interlock between the coating material 240 and the stent substrate 210. In some embodiments, the coating material can include more thin one polymer or more than one polymeric material. In certain embodiments, the average peel strength of the coating material can be within the ranges described above in connection with the embodiment of FIG. 1. Moreover, in certain embodiments, the average pore size can be within the ranges discussed above.

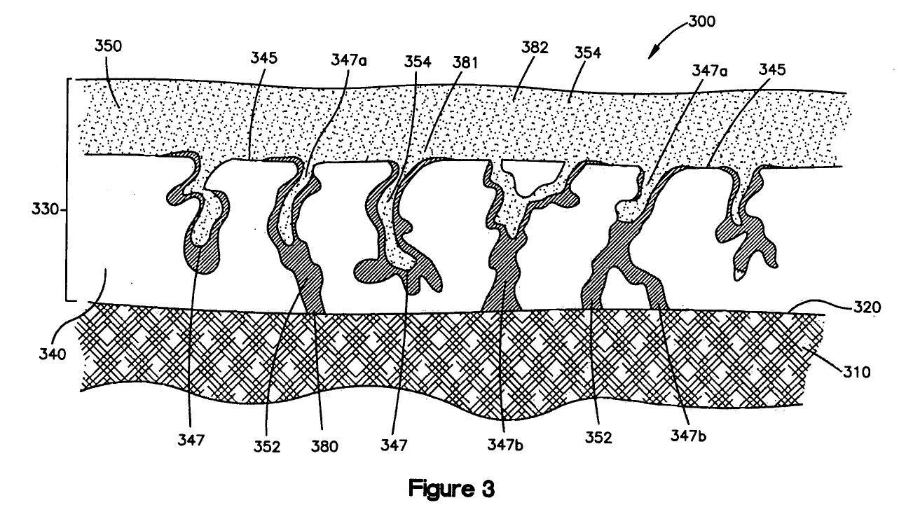

FIG. 3 shows a cross-sectional view of another embodiment of a coating disposed on a surface of a medical device, such as an intravascular stent. The coating in this embodiment includes a porous first coating material disposed on the surface of a medical device and a second coating material, comprising first and second coating compositions, disposed on the porous first coating material. The medical device 300 has a substrate 310 having a surface 320. A coating 330, which comprises a first coating material 340 and a second coating material 350, is disposed on at least a portion of the substrate surface 320. The first coating material 340 has a surface 345 and a plurality of pores 347 in the first coating material 340. At least some of the pores 347a are in fluid communication with the surface of the first coating material 345. The first coating material 340 can include a metal or a metal-containing compound such as a metal carbide, a metal nitride or a metal oxide. The second coating material 350 is disposed on at least a portion of the first coating material 340 and within at least one of the pores 347. The second coating material 350 can completely or partially fill the pores 347. The second coating material 350 comprises a first composition 352 and a second composition 354. The first composition 352 comprises a first polymer 380 and is substantially free of a therapeutic agent. The second composition 354 comprises a second polymer 381 and a therapeutic agent 382. In some embodiments, the first composition can be free of the therapeutic agent 382. In this embodiment, the presence of the second coating material 350 on the first coating material 340 and in the pores 347 forms an interlock between the second coating material 350 and the stent substrate 320. In some embodiments, at least some of the pores 347b extend from the first coating material surface 345 to the substrate surface 320, and the second coating material 350 in at least some of the pores 347b contacts the substrate surface 320.

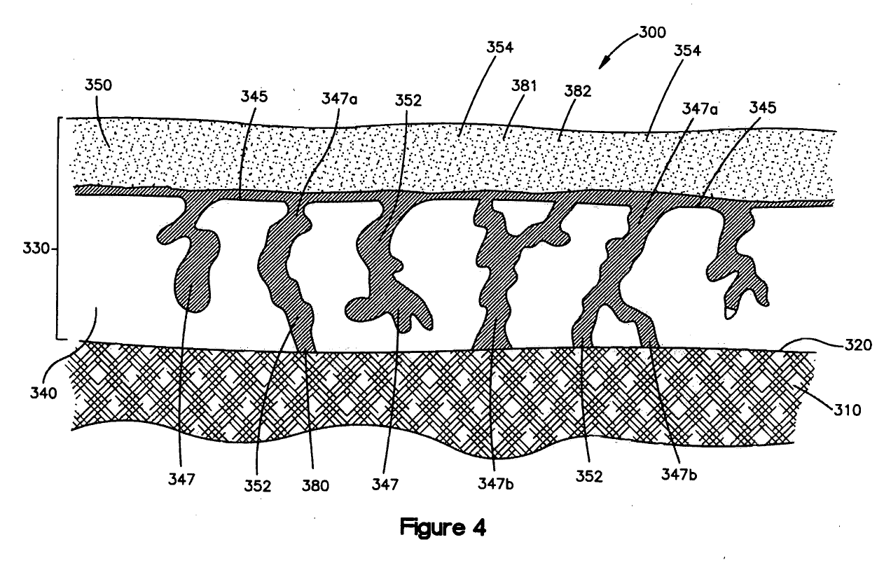

In the embodiment shown in FIG. 3, both the first composition 352 and the second composition 354 of the second coating material 350 are disposed in at least some of the pores 347. In the embodiment shown in FIG. 4, the first composition 352 is disposed in a plurality of pores 347 in a manner such that the pores 347 are substantially free of the second composition 354. In this embodiment, the pores 347 are filled with the first composition 352 and the first composition 352 is disposed on at least a portion of the surface of the first coating material 345. In alternative embodiments, the second composition, which includes a therapeutic agent, can be disposed in a plurality of pores in a manner such that the pores are substantially free of the first composition.

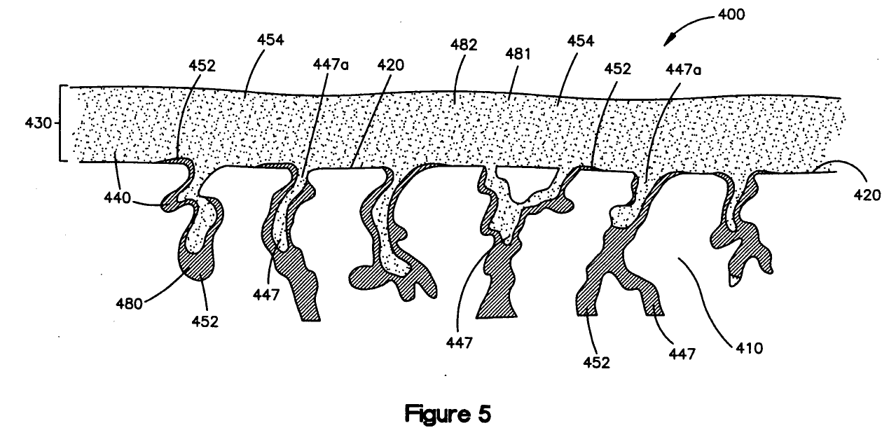

FIG. 5 shows a cross-sectional view of another embodiment of a coated medical device, such as a stent, that is similar to the embodiment shown in FIG. 2. In this embodiment, the substrate of the medical device comprises a plurality of pores. A coating comprising a coating material, which comprises first and second compositions, is disposed on the surface of the medical device and in the pores. More specifically, the medical device 400 has a substrate 410 and a surface 420. The substrate 410 comprises a plurality of pores 447. At least some of the pores 447a are in fluid communication with the substrate surface 420. A coating 430, which comprises a coating material 440, is disposed on the substrate surface 420 and in at least some of the pores 447. The substrate 410 can include a metal or a metal-containing compound such as a metal carbide, a metal nitride or a metal oxide. The coating material 440 comprises a first composition 452 and a second composition 454. The first composition 452 comprises a first polymer 480 and is substantially free of a therapeutic agent. The second composition 454 comprises a second polymer 481 and a therapeutic agent 482. In this embodiment, the presence of the coating material 440 on the substrate surface 420 and in the pores 447 forms an interlock between the coating material 440 and the stent substrate 410.

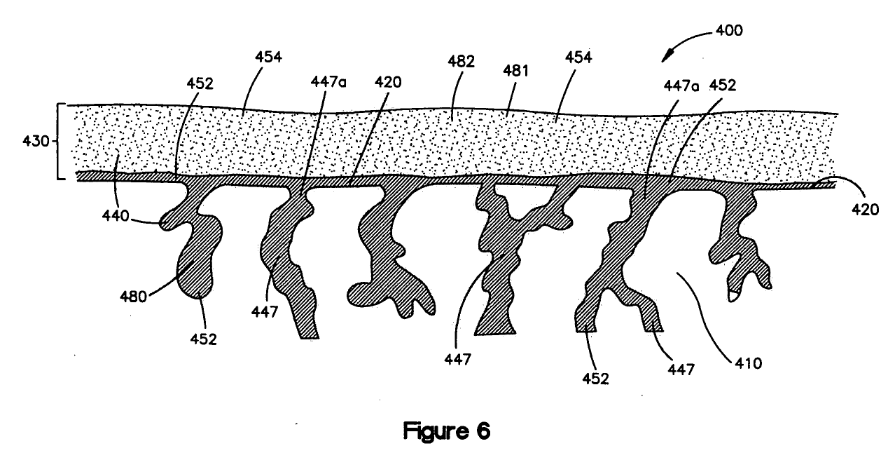

The embodiment shown in FIG. 6 is similar to that shown in FIG. 5. However, in FIG. 5, both the first composition 452 and the second composition 454 of the second coating material 430 are disposed in at least some of the pores 447. In contrast, in the embodiment shown in FIG. 6, the first composition 452 is disposed in a plurality of pores 447 in a manner such that the pores 447 are substantially free of the second composition 454. In this embodiment, the pores 447 are tilled with the first composition 452 and the first composition 452 is disposed on at least a portion of the substrate surface 420. In alternative embodiments, the second composition, which includes a therapeutic agent, can be disposed in a plurality of pores in a manner such that the pores are substantially free of the first composition.

### 6.2 Medical Devices

Suitable medical devices include, but are not limited to, stents, surgical staples, cochlear implants, catheters, such as central venous catheters and arterial catheters, guidewires, cannulas, cardiac pacemaker leads or lead tips, cardiac defibrillator leads or lead tips. implantable vascular access ports, blood storage bags, blood tubing, vascular or other grafts, intra-aortic balloon pumps, heart valves, cardiovascular sutures, total artificial hearts and ventricular assist pumps, extra-corporeal devices such as blood oxygenators, blood filters, hemodialysis units, hemoperfusion units or plasmapheresis units.

Medical devices which are particularly suitable for the embodiments described herein include any stent for medical purposes, which are known to the skilled artisan. Suitable stents include, for example, intravascular stents such as self-expanding stents and balloon expandable stents. Examples of self-expanding stents are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchasik et al. In preferred embodiments, a suitable stent is an Express stent. More preferably, the Express stent is an Express™ stent or an Express2™ stent (Boston Scientific, Inc., Natick, Mass.).

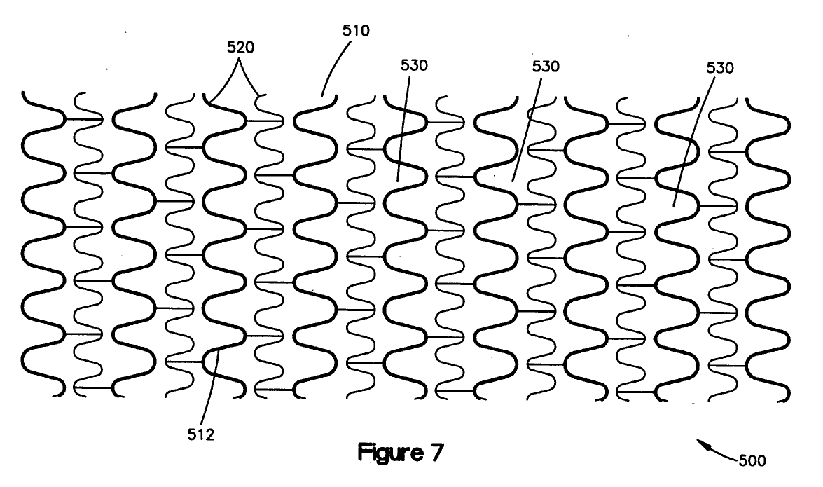

FIG. 7 shows an example of a stent that is suitable for use in the embodiments described herein. This figure shows an implantable intravascular stent 500 comprising a sidewall 510 which comprises a plurality of struts 520 and at least one opening 530 in the sidewall 510. Generally, the openings 530 are disposed between adjacent struts 520. Also, the sidewall 510 may have a first sidewall surface 512 and an opposing second sidewall surface, which is not shown in FIG. 7. The first sidewall surface 512 can be an outer or abluminal sidewall surface, which faces the body lumen surface when the stent is implanted, or an inner or luminal sidewall surface, which faces away from the body lumen surface and towards the lumen. Likewise, the second sidewall surface can be an abluminal sidewall surface or a luminal sidewall surface.

When the coatings are applied to a stent having openings in the stent sidewall structure, in certain embodiments, it is preferable that the coatings conform to the surface of the stent so that the openings in the sidewall stent structure are preserved, e.g. the openings are not entirely or partially occluded with coating material.

The stents may be formed through various methods as known in the art. The stents may be formed by welding, molding, laser cutting, or electro-forming. Also, the stents can be made by using filaments or fibers that are wound or braided together in order to form a continuous structure.

### 6.3 Medical Device Materials

Medical devices may be fabricated from metallic, ceramic, polymeric, non-polymeric or composite materials or a combination thereof. Preferably, the materials are biocompatible. Suitable metallic materials or metals include without limitation alkali metals, alkaline earth metals, transition metals, metal alloys and metalloids. Examples of metals include without limitation, titanium and titanium alloys (such as nitinol, nickel-titanium alloys, thermo-memory alloy materials), scandium, stainless steel (e.g., PERSS (Platinum EnRiched Stainless Steel)), tantalum, nickel, silicon, chrome, cobalt (e.g., cobalt chromium nickel alloys such as Elgiloy® and Phynox®), chromium, manganese, iron, platinum, iridium, niobium, vanadium, zirconium, tungsten, rhodium, ruthenium, gold, copper, zinc, yttrium, molybdenum, technetium, palladium, cadmium, hafnium, rhenium and combinations or alloys thereof. Metallic materials also include clad composite filaments, such as those disclosed in WO 94/16646. In some embodiments, the metal can be radiopaque and/or have MRI compatibility.

Suitable ceramic materials include, but are not limited to, metal oxides, carbides, or nitrides of the transition elements such as titanium, hafnium, iridium, chromium, aluminum, and zirconium. Silicon based materials, such as silica, may also be used. Suitable metal oxides that can be used include without limitation platinum oxides, tantalum oxides, titanium oxides, tantalum oxides, zinc oxides, iron oxides, magnesium oxides, aluminum oxides, iridium oxides, niobium oxides, zirconium oxides, tungsten oxides, rhodium oxides, ruthenium oxides, or combinations thereof. In some embodiments, the ceramic material can be radiopaque and/or have MRI compatibility.

Polymer(s) useful for forming medical devices should be ones that are biocompatible and avoid irritation to body tissue. The polymers can be biostable or bioabsorbable. Suitable polymers useful for making the substrate include, but are not limited to, isobutylene-based polymers, polystyrene-based polymers, polyacrylates, and polyacrylate derivatives, vinyl acetate-based polymers and its copolymers, polyurethane and its copolymers, silicone and its copolymers ethylene vinyl-acetate, polyethylene terephtalate, thermoplastic elastomers, polyvinyl chloride, polyolefins, cellilosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, chitins, or a combination thereof.

Other polymers that are useful as materials for making the substrate include, but are not limited to, dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(ÿ-caprolactone), poly(ÿ-hydroxybutyrate), polydioxanone, poly(ÿ-ethyl glutamate), polyiminocarbonates, poly(ortho ester), polyanhydrides, styrene isobutylene styrene, polyetheroxides, polyvinyl alcohol, polyglycolic acid, polylactic acid, polyamides, poly-2-hydroxy-butyrate, polycaprolactone, poly(lactic-co-clycolic)acid, Teflon, alginate, dextran, chitin, cotton, polyglycolic acid, polyurethane, derivatized versions thereof, (i.e., polymers which have been modified to include, for example, attachment sites or cross-linking groups, e.g., arginine-glycine-aspartic acid RGD, in which the polymers retain their structural integrity while allowing for attachment of cells and molecules, such as proteins and/or nucleic acids), or a combination thereof.

Furthermore, although in certain embodiments a single type of polymer is used to form the substrate; various combinations of polymers can also be employed. The appropriate mixture of polymers can be coordinated to produce desired effects when incorporated into a substrate.

### 6.4 Metallic Coating Materials

The coating materials of the medical devices described herein can include a metal or a metal-containing compound such as a metal carbide, metal nitride or a metal oxide. Suitable metals include without limitation alkali metals, alkaline earth metals, transition metals, metal alloys and metalloids. Examples of metals include without limitation, titanium and titanium alloys (such as nitinol, nickel-titanium alloys, thermo-memory alloy materials), scandium stainless steel (e.g., PERSS (Platinum EnRiched Stainless Steel)), tantalum, nickel, silicon, chronium, cobalt (e,g., cobalt chromium nickel alloys such as Elgiloy® and Phynox®), manganese, iron, platinum, iridium niobium, vanadium, zirconium, tungsten, rhodium. ruthenium, gold, copper, zinc, yttrium, molybdenum, technetium, palladium, cadmium, hafnium, rhenium and combinations or alloys thereof.

Suitable metal carbides and metal nitrides include, but are not limited to, carbides and nitrides of the metals listed above such as metal carbides and metal nitrides of transition elements such as titanium, hafnium, iridium, chromium, aluminum, and zirconium.

Suitable metal oxides that can be used as a coating material include, without limitation, oxides of the above metals. These include, without limitation, platinum oxides, tantalum oxides, titanium oxides, zinc oxides, iron oxides, magnesium oxides, aluminum oxides, iridium oxides, niobium oxides, zirconium oxides, tungsten oxides, rhodium oxides, ruthenium oxides, or combinations thereof.

The metal or a metal-containing compound of the coating material can be radiopaque and/or have MRI compatibility. Also, the coating materials can include the same or some of the same materials that are used to make the medical device.

### 6.5 Polymeric Coating Materials

Polymers useful in the coatings described herein should be ones that are biocompatible, particularly during insertion or implantation of the device into the body and avoids irritation to body tissue. Examples of such polymers include, but not limited to, polyurethanes, polyisobutylene and its copolymers, silicones, and polyesters. Other suitable polymers include polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate; copolymers of vinyl monomers, copolymers of vinyl monomers and olefins such as ethylene-methyl methacrylate copolymers, acrylonitrile-styrene copolymers. ABS resins, ethylene-vinyl acetate copolymers, polyamides such as Nylon 66 and polycaprolactone, alkyd resins, polycarbonates, polyoxyethylenes, polyimides, polyethers, epoxy resins, polyurethanes, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, and polylactic acid-polyethylene oxide copolymers.

In certain embodiment hydrophobic polymers can be used. Examples of suitable hydrophobic polymers or monomers include, but not limited to, polyolefins, such as polyethylene, polypropylene, poly(1-butene), poly(2-butene), poly(1-pentene) poly(2-pentene), poly(3-methyl-1-pentene), poly(4-methyl-1-pentene), poly(isoprene), poly(4-methyl-1-pentene), ethylene-propylene copolymers, ethylene-propylene-hexadiene copolymers, ethylene-vinyl acetate copolymers, blends of two or more polyolefins and random and block copolymers prepared from two or more different unsaturated monomers; styrene polymers, such as poly(styrene), poly(2-methylstyrene), styrene-acrylonitrile copolymers having less than about 20 mole-percent acrylonitrile, and styrene-2,2,3,3,-tetrafluoropropyl methacrylate copolymers; halogenated hydrocarbon polymers, such as poly(chlorotrifluoroethylene), chlorotrifluoroethylene-tetrafluoroethylene copolymers, poly(hexafluoropropylene), poly(tetrafluoroethylene), tetrafluoroethylene, tetrafluoroethylene-ethylene copolymers, poly(trifluoroethylene), poly(vinyl fluoride), and poly(vinylidene fluoride); vinyl polymers, such as poly(vinyl butyrate), poly(vinyl decanoate), poly(vinyl dodecanoate), poly(vinyl hexadecanoate), poly(vinyl hexanoate), poly(vinyl propionate), poly(vinyl octanoate), poly(heptafluoroisopropoxyethylene), poly(heptafluoroisopropoxypropylene), and poly(methacrylonitrile); acrylic polymers, such as poly(n-butyl acetate), poly(ethyl acrylate), poly(1-chlorodifluoromethyl)tetrafluoroethyl acrylate, poly di(chlorofluoromethyl)fluoromethyl acrylate, poly(1,1-dihydroheptafluorobutyl acrylate), poly(1,1-dihydropentafluoroisopropyl acrylate), poly(1,1-dihydropentadecafluorooctyl acrylate), poly(heptafluoroisopropyl acrylate), poly 5-(heptafluoroisopropoxy)pentyl acrylate, poly 11-(heptafluoroisopropoxy)undecyl acrylate, poly 2-(heptafluoropropoxy)ethyl acrylate, and poly(nonafluoroisobutyl acrylate); methacrylic polymers, such as poly(benzyl methacrylate), poly(n-butyl methacrylate), poly(isobutyl methacrylate), poly(t-butyl methacrylate), poly(t-butylaminoethyl methacrylate), poly(dodecyl methacrylate), poly(ethyl methacrylate), poly(2-ethylhexyl methacrylate), poly(n-hexyl methacrylate), poly(phenyl methacrylate), poly(n-propyl methacrylate), poly(octadecyl methacrylate ), poly(1,1-dihydropentadecafluorooctyl methacrylate), poly(heptafluoroisopropyl methacrylate), poly(heptadecafluorooctyl methacrylate), poly(1-hydrotetrafluoroethyl methacrylate), poly(1,1-dihydrotetrafluoropropyl methacrylate), poly(1-hydrohexafluoroisopropyl methacrylate), and poly(t-nonafluorobutyl methacrylate), polyesters, such a poly(ethylene terephthalate) and poly(butylene terephthalate); condensation type polymers such as and polyurethanes and siloxane-urethane copolymers; polyorganosiloxanes, i.e., polymers characterized by repeating siloxane groups, represented by Ra SiO_{4-a/2}, where R is a monovalent substituted or unsubstituted hydrocarbon radical and the value of a is 1 or 2; and naturally occurring hydrophobic polymers such as rubber.

In alternative embodiments, hydrophilic polymers can be used. Examples of suitable hydrophilic polymers or monomers include, but not limited to; (meth)acrylic acid, or alkaline metal or ammonium salts thereof; (meth)acrylamide; (meth)acrylonitrile; those polymers to which unsaturated dibasic, such as maleic acid and fumaric acid or half esters of these unsaturated dibasic acids, or alkaline metal or ammonium salts of these dibasic adds or half esters, is added; those polymers to which unsaturated sulfonic, such as 2-acrylamido-2-methylpropanesulfonic, 2-(meth)acryloylethanesulfonic acid, or alkaline metal or ammonium salts thereof, is added; and 2-hydroxyethyl (meth)acrylate and 2-hydroxypropyl (meth)acrylate.

Polyvinyl alcohol is also an example of hydrophilic polymer. Polyvinyl alcohol may contain a plurality of hydrophilic groups such as hydroxyl, amido, carboxyl, amino, ammonium or sulfonyl (—SO₃). Hydrophilic polymers also include, but are not limited to, starch, polysaccharides and related cellulosic polymers; polyalkylene glycols and oxides such as the polyethylene oxides; polymerized ethylenically unsaturated carboxylic acids such as acrylic, mathacrylic and maleic acids and partial esters derived from these acids and polyhydric alcohols such as the alkylene glycols; homopolymers and copolymers derived from acrylamide; and homopolymers and copolymers of vinylpyrrolidone.

Additional suitable polymers include, but are not limited to, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, polyether block amides, epoxy, resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, poly lactic acid, polyglycolic acid, polylactic acid-polyethylene oxide copolymers, EPDM (ethylene-propylene-diene) rubbers, fluoropolymers, fluorosilicones, polyethylene glycol, polysaccharides, phospholipids, and combinations of the foregoing.

In certain embodiments preferred polymers include, but are not limited to polyactic acid, polyglycolic acid, polylactic-glycolic acid, styrene-isobutylene-styrene block copolymers styrene-maleic anhydride random copolymer or combinations thereof.

### 6.6 Therapeutic Agents

The term “therapeutic agent” as used herein encompasses drugs, genetic materials, and biological materials and can be used interchangeably with “biologically active material.” The term “genetic materials” means DNA or RNA, including, without limitation, DNA/RNA encoding a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g. BMP-2, BMP-3, BMP-4, BMP-5, BMP-6 (Vgr-1). BMP-7 (PO-1), BMP-8, BMP-9, BMP-10, BMP-11, BMP-12, BMP-14, BMP-15. BMP-16. etc.), matrix metalloproteinase (TIMP), tissue inhibitor of matrix metalloproteinase (TIMP), cytokines, interleukin (e.g. IL-1, IL2, IL-3, IL-4, IL-5, IL-6, IL-7, IL-8, IL-9, IL-10, IL-11, IL-12, IL-15, etc.), lymphokines, interferon, integrin, collagen (all types) elastin, fibrillins, fibronectin, vitronectin, laminin, glycosaminoglycans, proteoglycans, transferrin, cytotactin, cell binding domains (e.g., RGD) and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogencic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other suitable therapeutic agents include:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, pimecrolimus, sirolimus, zotarolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives, paclitaxel as well as its derivatives, analogs or paclitaxel bound to proteins, e.g. Abraxane™;
anesthetic agents such as lidocaine, bupivacaine, and ropivacaine;
anti-coagulants Such as D-Phe-Pro-Arg chloromethyl ketone, an RGD peptide-containing compound, heparin antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine hirudin, prostaglandin inhibitors, platelet inhibitors antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxacillin, tobranycin, daunomycin, mitocycin;
angiogenic substances, such as acidic and basic fibroblast growth factors, estrogen including estradiol (E2), estriol (E3) and 17-beta estradiol;
drugs for heart failure, such as digoxin, beta-blockers, angiotensin-converting enzyme (ACE) inhibitors including captopril and enalopril, statins and related compounds;
macrolides such as sirolimus (rapamycin) or everolimus; and
AGE-breakers including alagebrium chloride (ALT-711).

Other therapeutic agents include nitroglycerin, nitrous oxides, nitric oxides, antibiotics, aspirins, digitalis, estrogen estradiol and glycosides. Preferred therapeutic agents include anti-proliferative drugs such as steroids, vitamins, and restenosis-inhibiting agents. Preferred restenosis-inhibiting agents include microtubule stabilizing agents such as Taxol®, paclitaxel (i.e. paclitaxel, paclitaxel analogs, or paclitaxel derivatives, and mixtures thereof). For example, derivatives suitable for use in the embodiments described herein include 2′-succinyl-taxol, 2′-succinyl-taxol triethanolamine, 2′-glutaryl-taxol, 2′-glutaryl-taxol triethanolamine salt, 2°-O-ester with N-(dimethylaminoethyl) glutamine, and 2′-O-ester with N-(dimethylaminoethyl) glutamine hydrochloride salt.

Other preferred therapeutic agents include tacrolimus: halofuginone; inhibitors of HSP90 heat shock proteins such as geldanamycin; microtubule stabilizing agents such as epothilone D; phosphodiesterase inhibitors such as cliostazole; Barket inhibitors; phospholamban inhibitors; and Serca 2 gene/proteins. In yet another preferred embodiment, the therapeutic agent is an antibiotic such as erythromycin, amphotericin, rapamycin, adriamycin, etc.

In preferred embodiments, the therapeutic agent comprises daunomycin, mitocycin, dexamethasone, everolimus, tacrolimus, zotarolimus, heparin, aspirin, warfarin, ticlopidine, salsalate, diflunisal, ibuprofen, ketoprofen, nabumetone, prioxicam, naproxen, diclofenac, indomethacin, sulindac, tolmetin, etodolac, ketorolac, oxaprozin, celcoxib, alagebrium chloride or a combination thereof.

The therapeutic agents can be synthesized by methods well known to one skilled in the art. Alternatively, the therapeutic agents can be purchased from chemical and pharmaceutical companies.

### 6.7 Methods of Making the Medical Devices

Provided herein are methods of making the medical devices described above. In certain embodiments, the coated medical devices, such as intravascular stents, can be made by providing a medical device or stent that comprises a substrate having a surface. A coating is formed on the substrate surface by disposing a first coating material on at least a portion of the substrate surface to form a first coating material surface. The first coating material comprises a metal or a metal-containing compound such as a metal carbide, a metal nitride or a metal oxide. A plurality of pores is formed in the first coating material. Preferably, at least some of the pores are in fluid communication with the first coating material surface. Also, preferably at least some of the pores are in fluid communication with the substrate surface. Also, the plurality of pores can be formed after the first coating material is disposed on the substrate surface. Furthermore, a second coating material is disposed on at least a portion of the first coating material surface and in at least some of the pores to form an interlock between the second coating material and the substrate. The second coating material comprises a first polymer and a first therapeutic agent. The average peel strength of the second coating material from the stent, in certain embodiments, is about 1000 grams per inch width or greater. In other embodiments the average peel strength is about 1000 grams per inch width to about 3000 grams per inch width.

In another embodiment, the method for making a coated medical device includes providing a medical device comprising a substrate having a surface. The substrate of the medical device can include a metal or a metal-containing compound such as a metal carbide, a metal nitride or a metal oxide having a plurality of pores therein. Preferably, some of the pores are in fluid communication with the substrate surface. A coating is formed on the substrate surface by disposing a coating material on at least a portion of the substrate surface and in at least some of the pores to form an interlock between the coating material and the substrate. The coating material comprises a first polymer and a first therapeutic agent. The average peel strength of the second coating material from the stent can be greater than about 250 grams per inch width; greater than about 500 grams per inch width; greater than about 750 grams per inch width; or greater than about 1000 grams per inch width. For example, the average peel strength can be about 250 grams per inch width to about 3000 grams per inch width; about 500 grams per inch width to about 3000 grams per inch width; or about 1000 grams per inch width to about 3000 grams per inch width.

Also, in another embodiment, the method for making an implantable coated medical device includes providing a medical device comprising a substrate having a surface. A coating is formed on the substrate surface by disposing a first coating material on at least a portion of the substrate surface to form a first coating material surface. The first coating material can include a metal or a metal-containing compound such as a metal carbide, a metal nitride or a metal oxide. A plurality of pores is formed in the first coating material. Preferably, at least some of the pores are in fluid communication with the first coating material surface. Also, preferably at least some of the pores are in fluid communication with the substrate surface. The pores can be formed after the first coating material is disposed on the substrate surface. A second coating material is disposed on at least a portion of the first coating material surface and in at least some of the pores to form an interlock between the second coating material and the substrate. The second coating material is disposed on the first coating material and in the pores by disposing a first composition that comprises a first polymer and that is substantially free of a therapeutic agent. Thereafter, a second composition that comprises a second polymer and a therapeutic agent is disposed on at least a part of the first composition. In some embodiments, the viscosity of the first composition is less than the viscosity of the second composition. Also, in some embodiments, the first and second polymers are the same.

In yet another embodiment of a method for making an implantable coated medical device, the method comprises providing an implantable stent comprising a substrate having a surface. The substrate comprises a metal or a metal-containing compound such as a metal carbide, a metal nitride or a metal oxide having a plurality of pores therein. At least some of the pores are in fluid communication with the substrate surface. A coating is formed on the substrate surface by disposing a coating material on at least a portion of the substrate surface and in at least some of the pores to form an interlock between the coating material and the substrate. The coating material is disposed on the substrate surface and in the pores by disposing a first composition that comprises a first polymer and that is substantially free of a therapeutic agent followed by disposing a second composition that comprises a second polymer and a therapeutic agent. In some embodiments, the viscosity of the first composition is less than the viscosity of the second composition. Also, in some embodiments, the first and second polymers are the same.

### 6.7.1 Preparing a Substrate or Coating Material Having a Plurality of Pores therein

The pores of the substrate can be created by any method known to one skilled in the art including, but not limited to, sintering, co-deposition, micro-roughing, laser ablation, drilling, chemical etching or a combination thereof. For example, the porous structure can be made by a deposition process such as sputtering with adjustments to the deposition condition, by micro-roughening using reactive plasmas, by ion bombardment, electrolyte etching, or a combination thereof. Other methods include, but are not limited to, alloy plating, physical vapor deposition, chemical vapor deposition sintering, or a combination thereof.

Additionally, the pores can be formed by removing a secondary material from the metal or a metal-containing compound used to form the substrate. In particular, the substrate is formed from a composition containing the metal or a metal-containing compound and the secondary material. The secondary material is then removed. Techniques for removing a secondary material include, but are not limited to, dealloying or anodization processes, or by baking or heating to remove the secondary material. The secondary material can be any material so long as it can be removed from the metal or a metal-containing compound. For example the secondary material can be more electrochemically active than the metal or a metal-containing compound. See published U.S. Application No. 2005/0266040.

The pores in the coating material that comprises a metal or a metal-containing compound can be created by any method known to one skilled in the art including, but not limited to, the ones discussed above in connection with the formation of pores in the metals or a metal-containing compounds used to form the substrate. For example, the pores can be formed by removing a secondary material from the metal or a metal-containing compound in the coating material. In particular, the coating material includes a metal or a metal-containing compound and a secondary material. After the coating material is applied to the substrate or another coating material, the secondary material is removed to form pores in the metal or a metal-containing compound. In other embodiments, the pores can be formed when the metal oxide is applied to the surface of the medical device or another coating composition.

### 6.7.2 Application of Coating Materials or Compositions

The coating materials or compositions are preferably formed by applying a solution or suspension that contains the desired constituents. For instance, to form a coating material that contains a metal or a metal-containing compound, such metal or a metal-containing compound can be dissolved or suspended in a solvent.

Also, where the coating material or compositions comprise a polymer or therapeutic agent, these constituents can be dissolved or suspended in a solvent. Suitable solvents include without limitation methanol, water, acetone, ethanol, butanone, and THF. The viscosity of the coating materials or compositions can vary depending on the methods of application used to apply the coating materials or compositions to the medical device. For example, spray application of a coating material or composition requires low viscosity coating material or coating composition, while knife coating requires a higher viscosity coating material or coating composition. The viscosity of coating materials or compositions containing a polymer can be from about 1 cps to about 10.000 cps, about 10 cps to about 10,000 cps, about 50 cps to about 10,000 cps, about 100 cps to about 10,000 cps, about 250 cps to about 10,000 cps, about 500 cps to about 10,000 cps, about 1,000 cps to about 10,000 cps, about 1 cps to about 5,000 cps, about 1 cps to about 3,000 cps, about 1 cps to about 2,000 cps, or about 1 to about 1000 cps.

The solutions or suspensions can be applied by any method known to one skilled in the art, including, but not limited to, dipping; spraying, such as by conventional nozzle or ultrasonic nozzle; knife coating; laminating; pressing; brushing; swabbing; rolling; electrostatic deposition; painting; electroplating; evaporation; plasma-vapor deposition; batch processes such as air suspension, pan coating or ultrasonic mist spraying; cathodic-arc deposition; sputtering, ion implantation; and all modern chemical ways of immobilization of molecules to surfaces, or a combination thereof. Preferably, the coating composition is applied by spraying, dipping, laminating, pressing, or a combination thereof.

In embodiments where the coating material is disposed in the pores, such materials can be disposed in the pores by any method known to one skilled in the art including, but not limited to, dipping, spray coating, spin coating, plasma deposition, condensation, electrochemical or, electrostatic methods, evaporation, plasma vapor deposition, cathodic arc deposition, sputtering, ion implantation, use of a fluidized bed, or a combination thereof.

### 7.0 EXAMPLES

### 7.1 Example 1

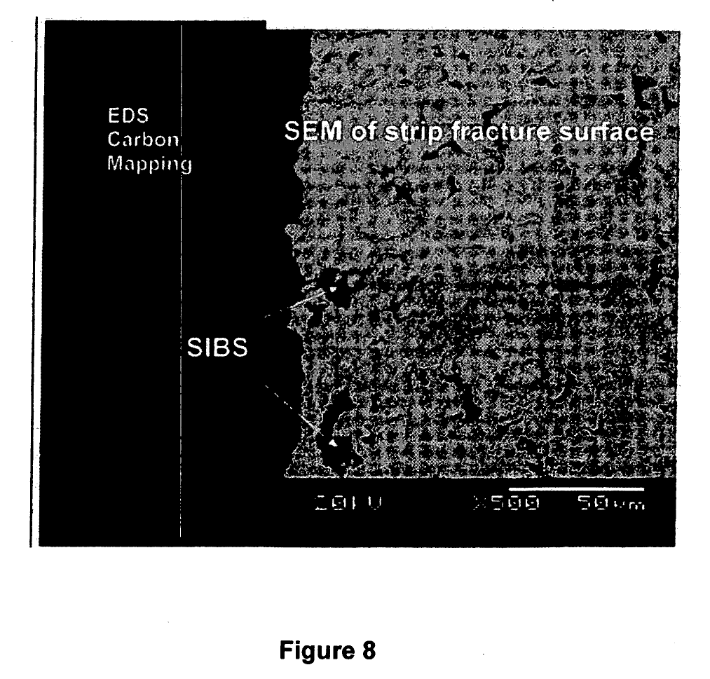

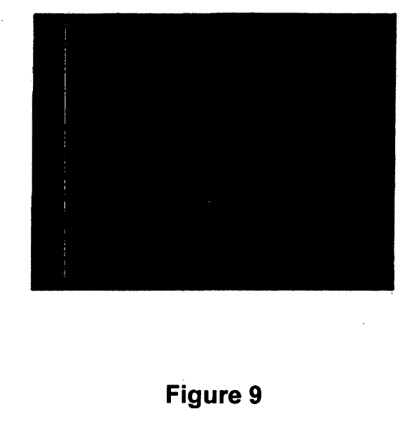

Polymer solutions containing either 1% or 3% by weight styrene-isobutylene-styrene block copolymer were sprayed onto porous stainless steel substrates containing pores having an average pore diameter of 0.2 μm. After the coatings were allowed to dry, cryo-fractured sections of the porous substrates were examined with scanning electron microscopy. FIG. 8 shows a scanning electron micrograph with mapping of the polymer carbon (in black) by an energy-dispersive spectrometer. As indicated by the white arrows, the polymer substantially fills the pores near the surface and penetrates to a depth greater than 30 microns (μm). FIG. 9 shows a scanning electron micrograph showing residual polymer (white) remaining attached to the porous substrates after removal of the coating. The polymer coating was found to remain bound within the 0.2 μm porous substrate surfaces after the polymer coating was peeled off, indicating good adhesion of the polymer coating into the porous surface.

### 7.2 Example 2

Polymer solutions containing 1%, 10%, and 25% solid by Meight styrene-isobutylene-styrene block copolymer were prepared. The polymer solutions were applied onto porous stainless substrates of either 0.2 μm or 1.0 82 m pore size via spray coating or knife blade coating (pouring coating fluid onto the substrate and spreading the fluid with a knife blade to a constant thickness). For the knife blade coating, a 25% solid by weight polymer solution was applied. Also a multi-step knife blade coating process was employed by applying a (1) 10% solid by weight polymer solution followed by a 25% solid by weight polymer solution; or (2) a 1% solid by weight polymer solution followed by a 25% solid by weight polymer solution.

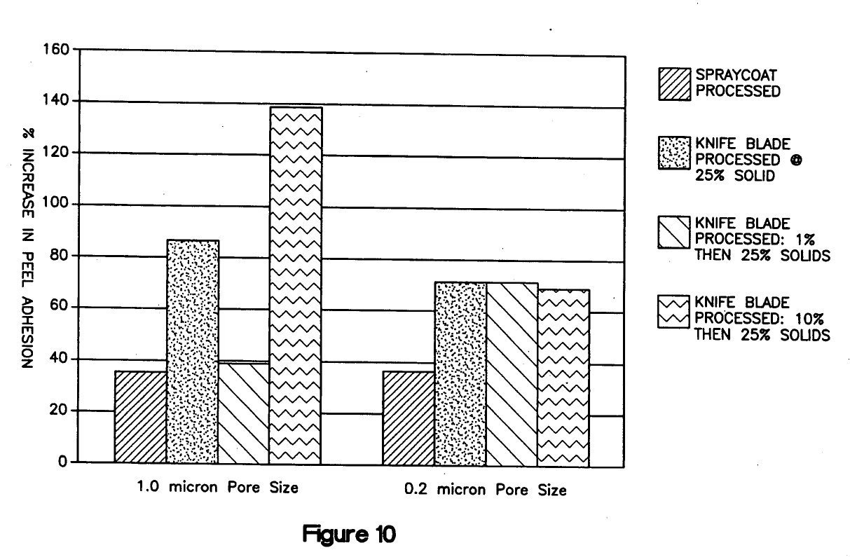

The percent increase in peel adhesion for each coating is shown in FIG. 10. Test pieces were about 0.5 inch wide, but could have been smaller. To compare, the measured forces were divided by the width of the coating strip in order to normalize the results.

For the substrate having 1.0 μm pores, the best adhesion compound to a milled finish control occurred when a lower viscosity coating solution (10% solids) was first applied by knife coating followed by a knife coating of a higher viscosity solution (25% solids). For the 0.2 μm substrate, the best adhesion occurred when the coating composition was applied by knife coating onto the substrate using either a single or multi-step knife coating process. Although not wishing to be bound by theory, this application order is thought to result is better penetration into the pore structure (via the low viscosity solution) and more complete pore filling (via the high viscosity and high solid content solution).

The foregoing description and examples have been set forth merely for illustration. Each of the disclosed aspects and embodiments described herein may be considered individually or in combination with other aspects, embodiments, and variations described herein. In addition, unless otherwise specified, none of the steps of the methods are confined to any particular order of performance. Modifications of the disclosed embodiments may occur to persons skilled in the art and such modifications are contemplated. Furthermore, all references cited herein are incorporated by reference in their entirety for all purposes.

We claim:

1. An implantable stent comprising:
(a) a substrate having a surface; and
(b) a coating disposed on at least a portion of the surface comprising:
(i) a first coating material having a first coating material surface and comprising a metal or a metal-containing compound having a plurality of pores therein, herein at least some of the pores are in fluid communication with the first coating material surface; and
(ii) a second coating material disposed on at least a portion of the first coating material surface and in at least some of the pores, forming an interlock between the second coating material and the substrate, wherein the second coating material comprises a first polymer and a first therapeutic agent; and
wherein the average peel strength of the second coating material from the stent is greater than about 1000 grams per inch width.

2. The stent of claim 1, wherein the average peel strength of the second coating material from the stent is about 1000 grams per inch width to about 3000 grams per inch width.

3. The stent of claim 1, wherein at least some of the pores extend from the first coating material surface to the substrate surface and the second coating material disposed in at least some of the pores contacts the substrate surface.

4. The stent of claim 1, wherein the pores have an average pore size of about 0.01 μm to about 10 μm.

5. The stent of claim 1, wherein the metal-containing compound is a metal oxide.

6. The stent of claim 1, wherein the therapeutic agent comprises an anti-thrombogenic agent, anti-angiogenesis agent, anti-proliferative agent antibiotic anti-restenosis agent growth factor, immunosuppressant or radiochemical.

7. The stent of claim 1, wherein the therapeutic agent comprises an agent that inhibits smooth muscle cell proliferation.

8. The stent of claim 1, wherein the first therapeutic agent comprises paclitaxel.

9. The stent of claim 1, wherein the first therapeutic agent comprises sirolimus, tacrolimus, pimecrolimus, zotarolimus or everolimus.

10. An implantable stent comprising:
(a) a substrate having a surface, wherein the substrate comprises a metal or a metal-containing compound having a plurality of pores therein, and wherein at least some of the pores are in fluid communication with the surface; and
(b) a coating comprising a coating material disposed on at least a portion of the substrate surface and in at least some of the pores, forming an interlock between the coating material and the substrate, wherein the coating material comprises a first polymer and a first therapeutic agent; and
wherein the average peel strength of the coating material from the stent is greater than about 1000 grams per inch width.

11. The stent of claim 10, wherein the average peel strength of the coating material from the stent is about 1000 grams per inch width to about 3000 grams per inch width

12. The stent of claim 10, wherein the pores have an average pore size of about 0.01 μm to about 10 μm.

13. The stent of claim 10, wherein the metal-containing compound is a metal oxide.

14. The stent of claim 10, wherein the first polymer is biostable.

15. The stent of claim 10, wherein the therapeutic agent comprises an anti-thrombogenic agent, anti-angiogenesis agent, anti-proliferative agent, antibiotic, anti-restenosis agent growth factor, immunosuppressant or radiochemical.

16. The stent of claim 10, wherein the first therapeutic agent comprises an agent that inhibits smooth muscle cell proliferation.

17. The stent of claim 10, wherein the first therapeutic agent comprises paclitaxel.

18. The stent of claim 10, wherein the first therapeutic agent comprises sirolimus, tacrolimus, pimecrolimus, zotarolimus or everolimus.

19. An implantable stent comprising:
(a) a substrate having a surface; and
(b) a coating disposed on at least a portion of the surface comprising:
(i) a first coating material having a surface and comprising a metal or a metal-containing compound having a plurality of pores therein, wherein at least some of the pores are in fluid communication with the first coating material surface; and
(ii) a second coating material disposed on at least a portion of the first coating material surface and in at least some of the pores, forming an interlock between the second coating material and the substrate, wherein the second coating material comprises a first composition that comprises a first polymer and that is substantially free of a therapeutic agent and a second composition that comprises a second polymer and a therapeutic agent.

20. The stent of claim 19, wherein the average peel strength of the second coating material from the stent is about 1000 grams per inch width to about 3000 grams per inch width

21. The stent of claim 19, wherein the average peel strength of the second coating material from the stent is greater than about 1000 grams per inch width.

22. The stent of claim 19, wherein at least some of the pores extend from the first coating material surface to the substrate surface and the second coating material disposed in at least some of the pores contacts the substrate surface.

23. The stent of claim 19, wherein both the first composition and the second composition are disposed in at least some of the pores.

24. The stent of claim 19, wherein the first composition is disposed in at least a plurality of the pores in a manner such that the pores are substantially free of the second composition.

25. The stent of claim 19, wherein the pores have an average pore size of about 0.01 μm to about 10 μm.

26. The stent of claim 19, wherein the metal-containing compound is a metal oxide.

27. The stent of claim 19, wherein the first polymer and second polymer are the same.

28. The stent of claim 19, wherein the therapeutic agent comprises an anti-thrombogenic agent, anti-angiogenesis agent, anti-proliferative agent, antibiotic, anti-restenosis agent, growth factor, immunosuppressant or radiochemical.

29. The stent of claim 19, wherein the therapeutic agent comprises an agent that inhibits smooth muscle cell proliferation.

30. The stent of claim 19, wherein the therapeutic agent comprises paclitaxel.

31. The stent of claim 19, wherein the therapeutic agent comprises sirolimus, tacrolimus, pimecrolimus, zotarolimus or everolimus.

32. An implantable stent comprising:
(a) a substrate having a surface, wherein the substrate comprises a metal or a metal-containing compound having a plurality of pores therein, and wherein at least some of the pores are in fluid communication with the surface; and
(b) a coating comprising a coating material disposed on at least a portion of the substrate surface and in at least some of the pores, forming an interlock between the coating material and the substrate, wherein the coating material comprises a first composition that comprises a first polymer and that is substantially free of a therapeutic agent, and a second composition that comprises a second polymer and a therapeutic agent.

33. The stent of claim 32, wherein the average peel strength of the coating material from the stent is about 1000 grams per inch width to about 3000 grams per inch width.

34. The stent of claim 32, wherein the average peel strength of the coating material from the stent is greater than about 1000 grams per inch width.

35. The stent of claim 32, wherein both the first composition and the second composition are disposed in at least some of the pores.

36. The stent of claim 32, wherein the first composition is disposed in at least a plurality of the pores in a manner such that the pores are substantially free of the second composition.

37. The stent of claim 32, wherein the pores have an average pore size of about 0.01 μm to about 10 μm.

38. The stent of claim 32, wherein the metal-containing compound is a metal oxide.

39. The stent of claim 32, wherein the therapeutic agent comprises an anti-thrombogenic agent, anti-angiogenesis agent, anti-proliferative agent, antibiotic, anti-restenosis agent, growth factor, immunosuppressant or radiochemical.

40. The stent of claim 32, wherein the therapeutic agent comprises an agent that inhibits smooth muscle cell proliferation.

41. The stent of claim 32, wherein the therapeutic agent comprises paclitaxel.

42. The stent of claim 32, wherein the therapeutic agent comprises sirolimus, tacrolimus, pimecrolimus zotarolimus or everolimus.
